(19) United States
(12) Patent Application Publication
(10) Pub. No.: US 2025/0143601 A1
(43) Pub. Date: May 8, 2025

(54) A System And Method For Assessing A Mobility Indicator Of A Patient

(71) Applicant: Adapttech Prosthetics Limited, Solihull (GB)

(72) Inventors: Frederico Alberto Abreu Carpinteiro, Porto (PT); Ana Sofia Miranda De Castro Resende Assis, Porto (PT); Marina Neves Miranda De Castro, Porto (PT)

(21) Appl. No.: 18/685,013

(22) PCT Filed: Aug. 22, 2022
(86) PCT No.: PCT/EP2022/073300

§ 371 Date: Feb. 20, 2024

(30) Foreign Application Priority Data
Aug. 24, 2021 (LU) LU500568

(51) Int. Cl. A61B 5/11; A61B 5/00

(52) U.S. Cl. CPC A61B 5/112; A61B 5/6811

(57) ABSTRACT

A system (100) and method for monitoring the gait of an amputee for assessment of a mobility indicator which is a resultant of a combination of clinically relevant outputs. The system (100) comprises at least two monitors (20) which are in connection with each other and disposed on different positions on at least one of the prosthetic device (10) or the body part (50) of the amputee. The at least two monitors (20) obtain mobility data (25) of the amputee in daily life. The system (100) further comprises an assessment tool (30) for assessing the mobility indicator based on the obtained mobility data (25). A report tool (40) reports the mobility indicator to the amputee.

### CROSS-REFERENCE TO RELATED APPLICATIONS

This application claims benefit of and priority to Luxembourg patent application LU500568 filed on 24 Aug. 2021. The entire disclosure of LU500568 is hereby incorporated by reference.

### FIELD OF THE INVENTION

The field of the invention concerns prosthetic technology and more particularly to a system for an assessment of a lower limb patient mobility indicator and a method for assessing a mobility indicator of an amputee using a prosthetic device.

### BACKGROUND OF THE INVENTION

The assessment of a lower limb patient's mobility indicator is performed by clinicians (M.D.) with many years of amputee subspecialty experience. The assessment is based on quantitative data from different so-called “gold standard tests” done in-clinic which mostly does not represent the patient's environment and the challenges in daily life. The assessment is based on the personal experience of the clinician, and the collected set of data during the standard test. The current, most widely used test is the so-called AMP (amputee mobility predictor) test, which when performed in amputees with prosthesis can be referred to as AMPPRO and which was developed by Bob Gailey, and is accepted by the US Medicare programs (see https://www.physiopedia.com/Amputee_Mobility_Predictor—downloaded on 22 Apr. 2021)

The mobility indicator is indicative, for example, of a current level of the so-called “K-level” of a lower limb patient. The K-Levels are means to quantify the need and the potential benefit of prosthetic devices for patients after lower limb amputation. The K-levels describe the types of activities a patient can perform and ultimately determines the eligibility and coverage for certain lower extremity prosthetic components. There are five K-levels, from level zero to level four.

With level zero, the patient does not have the ability or potential to ambulate or transfer safely with or without assistance and a prosthetic device does not enhance their quality of life or mobility. This level does not warrant a prescription for a prosthetic device. With level one, the patient has the ability or potential to use a prosthetic device for transfers or ambulation on level surfaces at fixed cadence. This is typical of a household ambulator or a person who only walks about in their own home. With level two, the patient has the ability or potential for ambulation with the ability to traverse low-level environmental barriers such as curbs, stairs, or uneven surfaces. This is typical of the limited community ambulator. With level three, the patient has the ability or potential for ambulation with variable cadence. A person at level 3 is typically a community ambulator who also has the ability to traverse most environmental barriers and may have vocational, therapeutic or exercise activity that demands prosthetic use beyond simple locomotion. With level four, the patient has the ability or potential for prosthetic ambulation that exceeds basic ambulation skills, exhibiting high impact, stress, or energy levels. This is typical of the prosthetic demands of the child, active adult, or athlete.

The U.S. Pat. No. 8,998,829 B1 discloses a system to assess an amputee patient function. The U.S. Pat. No. 9,408,560 B2 discloses a system to assess an activity level of a user. Both patents comprise a pedometer that records the number of steps over a defined period of time and a moment sensor that records the moments experienced by a prosthetic device are used in a networked computer environment to assess the functional activity level and instability of the lower limb amputee patient. The networked environment may include a user computer and a server computer in communication through the Internet. Both the user computer and the server computer include a functional assessment tool and a stability assessment tool. The tools on the user computer and server computer cooperate in assessing the activity level and the instability of a lower limb amputee patient.

The U.S. Pat. No. 8,998,829 B1 disclose an activity monitor that has a sensor for sensing movement, a processor for processing sensed data and a memory, wherein the processor is configured to use the sensed data to determine the number of steps taken for each of a plurality of epochs and to determine a measure of the fraction of each epoch spent stepping, the monitor being configured to record in a long term part of the memory at least two of the number of steps; the measure of the fraction of each epoch spent stepping and a measure of cadence calculated using the number of steps and the fraction of each epoch spent stepping.

Based on the collected in-clinic set of data and the interpretation thereof by the clinicians, it is possible that an incorrect mobility indicator is attributed.

Further examples of sensor enabled tests are described in Raykov, Yordan P., et al. “Probabilistic modelling of gait for robust passive monitoring in daily life.” IEEE Journal of Biomedical and Health Informatics (2020). which uses sensors for gait pattern analysis (accelerometer) to detect freezing of gait. The authors talk about the concept of performing tests on patients based on data coming from Activities of Daily Life (ADLs) as passive monitoring.

Th Jallon, Pierre, Benjamin Dupre, and Michel Antonakios. “A graph-based method for timed up & go test qualification using inertial sensors.” 2011 IEEE International Conference on Acoustics, Speech and Signal Processing (ICASSP). IEEE, 2011. discuss automatically performing the TUGT (timed up and go test) to predict a patient's ability to go outside alone safely. This demonstrates that if every action in the test can be identified automatically and characterized, they can be done blindly during the day, as the patient transitions from standing to sitting and vice-versa.

The US patent application US 2018/098865 A1 describes an apparatus for achieving a best possible prosthetic fit in a prosthesis system for an amputee. The apparatus includes a sensor array embedded in a gel like substance slipped over a residual limb in a socket configuration with respect to a prosthesis system.

Bruinsma J, Carloni R. “IMU-Based Deep Neural Networks: Prediction of Locomotor and Transition Intentions of an Osseointegrated Transfemoral Amputee”. IEEE Trans Neural Syst Rehabil Eng. 2021 describe the design and comparison of deep neural networks for the real-time prediction of locomotor and transition intentions of an amputee using data from inertial measurement units. The inertial measurement units are placed on different locations of the prosthetic device of the amputee.

An objective of the present disclosure is to suggest a mobility indicator for lower limb amputee patients based on an assessment of the individual daily life challenges in the amputee's personal environment and to provide guidance on which aspects the patient should focus to improve the mobility indicator. Based on those factors a prosthetic device should match the patient's need and potential.

### BRIEF DESCRIPTION OF THE INVENTION

The present document describes a system for monitoring the gait of an amputee patient for assessment of patient functionality, also termed a mobility indicator. The system comprises at least two monitors which are in connection with each other and disposed on different positions on at least one of the prosthesis or the residual body part of the patient. The at least two monitors obtain mobility data about the mobility of the patient in daily life and the fitting of the prosthetic. The system further comprises an assessment tool for assessing the mobility indicator based on the obtained mobility data. The mobility indicator is a resultant of a combination of clinically relevant outputs. A report tool reports the mobility indicator to the amputee. The report tool reports the mobility indicator which is obtained from the mobility data instead of reporting only the mobility data.

In an aspect, the assessment tool can perform an amputee mobility predictor (AMPPRO) test. Details of the AMPPRO test are given in the introduction to this document.

In an aspect, the at least two monitors each comprise at least one of a pressure sensor, an inertial motion unit, a battery, a clock, a memory, barometer, and a wireless communication unit. With those sensors it is possible to obtain mobility data such as for example a number of steps, number of strides, balance, cadence, cadence variability, speed, foot clearance, stride length, stride duration, step length, step duration, stance duration, swing duration, walking detection, walking bouts detection, ramp detection, running detection, turning detection and stairs detection.

In an aspect, the at least one of the report tool and the assessment tool is adapted to run on one of a mobile phone, a personal computer, or a cloud-based system. Thus, the amputee gets the information of the actual mobility indicator as well as other information directly and in real time.

The present document also discloses a method for the assessment of patient functionality of the amputee patient using the prosthesis. The method comprises the steps of measurement of mobility data by at least two monitors, disposed on different positions on at least one of the prosthetic device or a body part of the amputee, analysis of the mobility data, and assessment of the mobility indicator from the analyzed mobility data. The mobility indicator is resultant of a combination of clinically relevant outputs. The method further comprises reporting the mobility indicator to a report tool.

### BRIEF DESCRIPTION OF THE FIGURES

A more complete appreciation of the invention and many of the attendant advantages thereof will be readily obtained as the same becomes better understood by reference to the following detailed description when considered in connection with the accompanying figures.

FIG. 1 is a conceptional view of a first aspect of a system as disclosed herein.

FIG. 2 is a conceptional view of a second aspect of the system as disclosed herein.

FIG. 3 is a flowchart of the method as described herein.

### DETAILED DESCRIPTION OF THE INVENTION

The invention will now be described based on the figures. It will be understood that the embodiments and aspects of the invention described herein are only examples and do not limit the protective scope of the claims in any way. The invention is defined by the claims and their equivalents. It will be understood that features of one aspect or embodiment of the invention can be combined with a feature of a different aspect or aspects and/or embodiments of the invention.

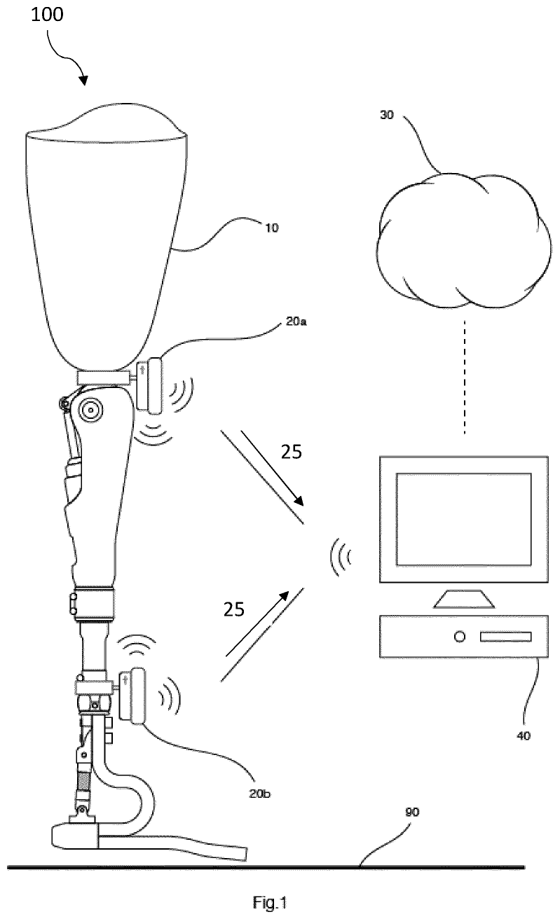

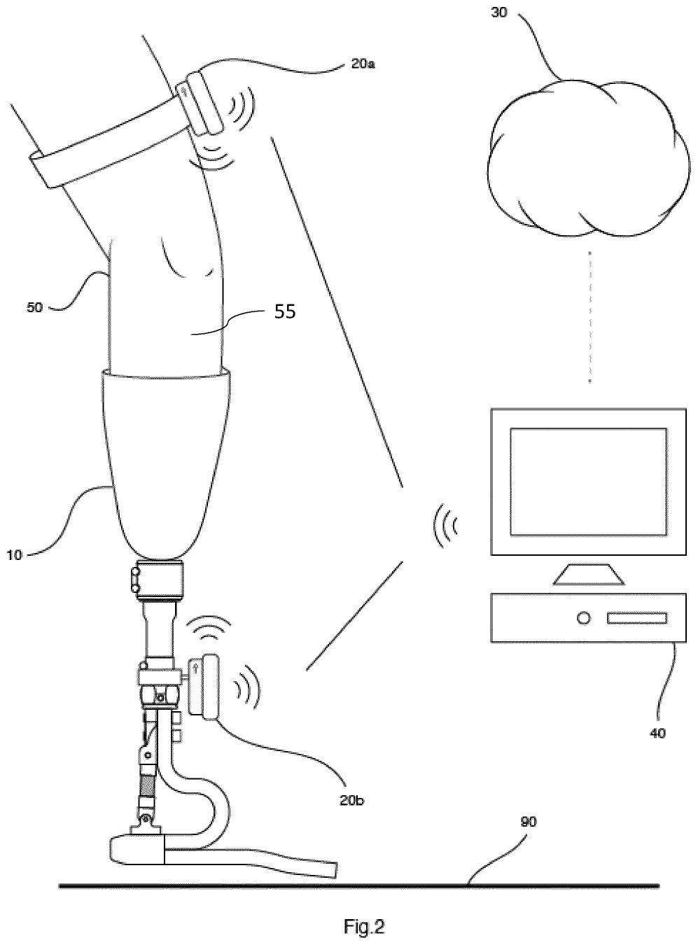

FIG. 1 show a first aspect of a system 100. The system 100 comprises a prosthesis 10 for a residual body part 50 of a patient 55. The prosthesis 10 of the first aspect, is a prosthetic device to accommodate a body part 50 such as a residual lower limb 55 (shown in FIG. 2). The system 100 further comprises at least two monitors 20a and 20b. The two monitors 20a and 20b communicate with each other wirelessly and independently with each to a mobile device, such as a smart phone, or a personal computer. The two monitors 20a and 20b are disposed on different positions on at least one of the prostheses 10 or the body part 50 of the amputee. For example, one monitor 20a is disposed at a first position of the prosthesis 10, for example at a position close to the body part 50 of the patient. The other monitor 20b is disposed at a second position of the prosthesis 10, for example close to a surface 90 on which the patient moves with the prosthesis 10. The two monitors 20a, 20b comprise various types of sensors. The sensors of the monitors 20a and 20b include one or more of a pressure sensor, an inertial motion unit, a battery, a clock, a memory, barometer, a magnetometer, a gyroscope, an accelerometer, and a wireless communication unit.

The two monitors 20a and 20b obtain mobility data 25 from the plurality of sensors noted above. The mobility data 25 are one of a number of steps, number of strides, balance, cadence, cadence variability, speed, foot clearance, stride length, stride duration, step length, step duration, stance duration, swing duration, walking detection, walking bouts detection, ramp detection, running detection, turning detection, stairs detection. The mobility data is gathered during the daily life of the amputee.

The system 100 further comprises an assessment tool 30. The assessment tool can perform an assessment of the mobility data 25 yielding a mobility indicator. This mobility indicator is a resultant of the combination of several clinically relevant outputs, such as an overview of the mobility data on a long-term acquisition, specific aspects of the mobility data (activity-related quantities that can be derived from sensor data), but also the results of using the data to perform automated clinical tests and getting blind outcome measures. The activity related quantities include, but are not limited to, number of steps, number of strides, balance, cadence, cadence variability, speed, foot clearance, stride length, stride duration, step length, step duration, stance duration, swing duration, walking detection, walking bouts detection, ramp detection, running detection, turning detection, stairs detection. The length of time for the long-term acquisition of the mobility data will depend on several factors. One example would be the frequency of the appointments with the patient.

The mobility indicator could be based on/similar to the Modus Trex score developed by Modus Inc which incorporates parameters such as measured cadence variability, energy expended during ambulation, the maximum activity measured and the K-level assessed by the clinician (source: Godfrey, Bradeigh, et al. “The accuracy and validity of modus Trex activity monitor in determining functional level in veterans with Transtibial amputations.” JPO: Journal of Prosthetics and Orthotics 30.1 (2018): 20-30).

The mobility indicator can further be based on the detection of specific activities relevant for the distinction of different K-levels as the case of detecting the patient negotiating curbs, using stairs, traversing inclines and declines and running.

Clinically relevant outputs can include any study-related activity that could have an effect, adverse or otherwise, on the clinical treatment of the patient. Clinically relevant further means any mobility data 25 and metric which is relevant towards distinguishing different k-levels, i.e. in describing the type of activities the patient performs.

The clinician sends the patient home with the system and has the patient send the device back or come back to the clinic, for example, after “sufficient data” is acquired. The period of acquisition would have a minimum and maximum threshold, of course, given the quantity of data needed to get the metrics, but also the storage capacity or method of data transferring.

The clinical tests results will be computed using the sensor-acquired mobility data to evaluate the performance of the patient automatically and objectively in a functional task of a clinical test, getting a certain outcome measure. Further data that does not relate directly to mobility and cannot be acquired through metrics resulting from data of the sensors of the monitors 20a and 20b will be input by the user in a textual or numerical manner. The sensor-acquired data is a raw input that can be processed for “human activity recognition”, transforming, for example, the data from accelerometers and gyroscopes into detected steps and strides that can be quantified and analyzed further. From these steps and strides it is possible to derive speed, variability, duration of each gait phase, like stance, swing, step length, presence of ramps or stairs . . . and these kinds of data are used to get results on clinical tests. This is because, for example, when the clinician performs an AMPPRO test on a patient, the clinician asks the patient to take some steps and analyze the continuity of walking bout. The mobility data acquired in the system would for example characterize the continuity of the walking bout, registering if there were any stops made by the patient. Another example in AMPPRO would be using the sensor-acquired data to calculate a measure of variable cadence, since one of the parameters in the AMPPRO test is the ability to vary cadence, having or not asymmetrical step lengths.

Of course, in the example of the AMPPRO test, there are some unanswerable questions answer, which the system of this document is not able to provide an answer, such as the sitting reach (it is not possible to monitor currently the patient reaching forward and grasping objects with the currently available sensors), or testing support with eyes close, as it is not possible see the eyes. Further developments of the system may enable these additional kinds of clinical tests and the current system provides a platform for their execution and automation and objective measures on the points we can quantify using the sensors.

The blind outcome measures are results of certain clinical tests that are acquired through the mobility data that is blindly retrieved, meaning that the patient does not perceive that the measure is being calculated, nor that their mobility data, at a certain moment in time, is being used as input for one of the Gold Standard Automated Clinical Tests, such as AMPPRO, 2MWT, 6MWT, and other tests. These tests intend to combine the autonomous, ubiquitous, and unobtrusive nature of wearable devices with the unsupervised and blind (without the patient's knowledge) testing methods for data acquisition. Using an unsupervised methodology reduces the white coat effect, increasing the patient's autonomy and eliminating the necessity of a technician carrying out the data acquisition, while still gathering patient information. Similarly, a blind methodology will allow the patient to perform their activities of daily living without the interference of test awareness and examiners. As an example of blindly acquiring data for the performance of a Clinical Test, one can utilize the mobility data of a two-minute walk to compute the 2 Minute Walking Test (2MWT) score. The system 100 further comprises a report tool 40. The report tool 40 reports the mobility indicator to the amputee. The reports that will be presented by this tool will centralize and combine traditional clinical tests' outcome measures, either acquired in a guided way or blindly, plus insights derived from long-term data acquisitions.

The report tool 40 can present as the results the mobility indicator and other metrics to both the patient and the prosthetist.

The report tool 40 can present the results in a user-friendly manner, either in a personal computer, a cloud-based system or a mobile device of the patient and/or the prosthetist. The report tool 40 can allow exporting via email or a document file (e.g. pdf). The report tool 40 will encompass the mobility metrics which, whenever possible, will be put in comparison with typical values for different k-levels and can be presented in different forms such as graphical or textual form. The variability of cadence, for example, can be shown as a graphic representing the number of steps taken by the patient per minute through the long-term acquisitions, contextualizing with reference thresholds.

The report tool 40 can adjust its contents to the target audience (patient or prosthetist).

It will be appreciated that the mobility indicator may not be a single value but could include a set of mobility metrics. To convey the mobility/ambulation performance of the patient, the patient's typical ambulation and best (ambulation) performance can be represented. Mobility metrics can therefore be represented as an average and the maximum or minimum value of a given type of mobility data 25.

The mobility metrics can include one or several of the following:

Average, variance and the maximum walk bout length and time. These can be determined from the detected walking bout (consecutive period of time the patient was walking consecutively) and speed.

The average, variance and maximum daily energy expenditure. These can be determined from the patient's weight and the daily number of strides the patient has performed.

The average, variance and maximum cadence and speed. These can be determined from the cadence and speed evidenced by the patient throughout the long-term acquisition.

The number of instances detected every day, the average, variance and the maximum covered distance. These can be determined from the blind 2 and 6 minute walk tests.

The total time spent doing each of those activities both relatively (e.g., 90% of this patient mobility was walking) and in time units (e.g. in 10 days of mobility data acquisition, this patient was able to transverse stairs and incline/declines for a 1 hour). These can be determined from the detected activities types.

The time doing each level of activity intensity either relatively, and in time units (similarly to the above point). These can be determined from the cadence evidenced by the patient throughout the long-term acquisition, segment time into periods of low, medium and high intensity of activity (similarly to Modus Inc, in the Stepwatch metrics (https://modushealth.com/wp-content/uploads/2020/10/SW4-metrics-RE-app-190323.pdf) (low=1 to 15, medium=16 to 40 and high=41 or greater steps of the prosthetic leg per minute).

The average, variance and maximum number of stairs traversed daily.

The two monitors 20 communicate wirelessly with at least one of the assessment tool 30 and the report tool 40. The assessment tool 30 and the report tool 40 are adapted to run on a device such as, but not limited to, the mobile device such as a mobile phone, the personal computer, or the cloud-based system. In one aspect, the assessment tool 30 and the report tool 40 run both on the same device. It will be understood that the assessment tool 30 and the report tool 40 can be combined into one application or program when run on one device.

FIG. 2 show a second aspect of the system 100. The two monitors 20a and 20b are disposed on different positions. For example, the monitor 20a is disposed at the body part 50 of the amputee, for example at a position close to the prosthetic device 10. For example, the monitor 20a is disposed at the residual body part 50 of the patient, for example at another anatomically relevant location such as, but not limited to, a shank, thigh, lower back near the sacrum bone. The other monitor 20b is disposed at the prosthetic device 20, for example close to the surface 90 on which the amputee moves with the prosthesis 10.

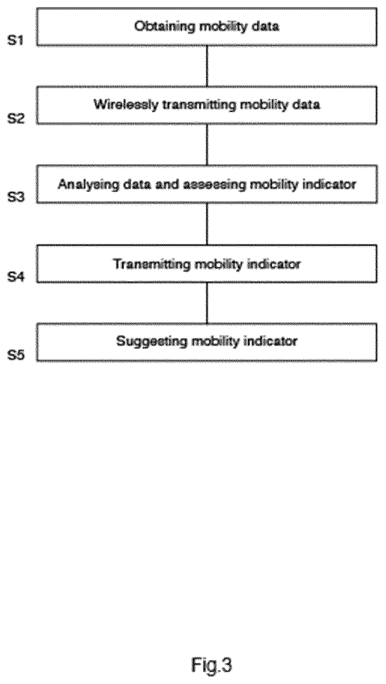

FIG. 3 show a method for assessing the mobility indicator of the patient using the prosthetic device.

In step S1 the mobility data 25 is obtained by the sensors of the two monitors 20. The obtained mobility data is then transmitted in step S2 wirelessly or through a wired connection to the assessment tool 30. In step S3 the assessment tool 30 performs an analysis of the raw sensor data received from the sensors and to produce an assessment of the mobility data yielding the mobility indicator.

In one non-limiting example, the mobility data 25 that is received from the sensors concerns accelerometer, gyroscope and magnetometer data that can be processed into meaningful biodata that relates to mobility. This means that raw sensor quantities received from the two monitors 20 are sufficient for the generation of processed information on mobility, as the number of steps, number of strides, balance, cadence, cadence variability, speed, foot clearance, stride length, stride duration, step length, step duration, stance duration, swing duration, walking detection, walking bouts detection, ramp detection, running detection, turning detection and stairs detection. This processed mobility data is then transmitted to the assessment tool 30, which then performs its analysis, yielding the referred mobility indicator.

The mobility indicator is a determined by a model that takes into account the processed mobility data. The assessment tool 30 uses a previously acquired training dataset of data of the same kind to decide on the mobility indicator for a present instance of data acquired, on a certain subject. This means that assessment tool 30 has learning capabilities that allow the assessment tool 30 to infer about the mobility indicator for a certain patient, following a certain data acquisition, based on analogous information retrieved previously, on the said training of the model, which characterizes different hypotheses of mobility indicators.

In step S4 the result of the assessment tool 30 is transmitted to the report tool 40. It will be appreciated that the assessment tool 30 and the report tool 40 could be incorporated into the same software running on the same device, such as an external computer or a smartphone. If the assessment tool 30 and the report tool 40 do not run on the same device, the result of the assessment is transmitted wirelessly (or by a connection) from the assessment tool 30 to the report tool 40. In step S5 a value for the mobility indicator is output by the report tool 40 to the amputee or another person, such as a medical practitioner or a physiotherapist.

The mobility indicator is indicative, for example, of a current level of the so-called “K-level”, a scale or a collection of relevant metrics and their relationships.

In one further aspect of the invention, training data can be used to develop a model of the amputee patient's mobility. With time, a database of mobility data is built with representative enough instances of the amputee population. The training data is obtained by clinicians monitoring the mobility of the patient and collecting clinical data. At the same time the sensor data is collected. Clinical data and mobility indicators that come from sensor data may provide diversified inputs: information regarding strides and their characteristics, along with activities of daily living quantification and qualification and mobility statistics, for example, may be integrated with information that comes from the analysis of a clinical specialist, hence the clinical data, that is equally input into the system. The clinical data and the sensor data are fed into a correlation system which correlates the clinical data with the sensor data and thereby trains the model. The model can be individualized and updated by adding further observations with individual ones of the patients and their prosthesis.

One example would be a patient climbing up the stairs. The sensor data will give the speed of both lateral and vertical movement. The two monitors 20 will provide the sensor data about uneven movement, such as between different legs, and indicate the difficulties that the patient has in walking up the stairs. The observations made by the clinician will provide further, more subjective, data about the patient's mobility. This data is fed into the model and can be used to assess automatically other patients to determine their degree of difficulty in climbing up stairs.

### REFERENCE NUMERALS

10 Prosthesis
20 Monitors
25 Mobility data
30 Assessment tool
40 Report tool
50 Residual body part
100 System

1. A system for monitoring the mobility of a patient for assessment of a mobility indicator, the system comprising:
a prosthesis for a residual body part of the patient;
at least two monitors in connection with each other, disposed on different positions on at least one of the prosthesis or the residual body part of the patient, for obtaining mobility data;
an assessment tool for performing an assessment of the mobility data yielding the mobility indicator which is a resultant of a combination of clinically relevant outputs; and
a report tool for reporting the mobility indicator.

2. The system according to claim 1, wherein the assessment tool performs a K-level assessment of the patient.

3. The system according to claim 1, wherein the at least two monitors each comprise at least one of a pressure sensor, an inertial motion unit, a battery, a clock, a memory, barometer, an accelerometer, a gyroscope, a magnetometer, and a wireless communication unit.

4. The system according to claim 1, wherein the at least two monitors communicate wirelessly with at least one of the report tool and the assessment tool.

5. The system according to claim 1, wherein the mobility data are one of a number of steps, number of strides, balance, cadence, cadence variability, speed, foot clearance, stride length, stride duration, step length, step duration, stance duration, swing duration, walking detection, walking bouts detection, ramp detection, running detection, turning detection, stairs detection.

6. The system according to claim 1, wherein the at least one of the report tool and the assessment tool is adapted to run on one of a mobile phone, a personal computer, or a cloud-based system.

7. A method for assessing a mobility indicator of an amputee using a prosthetic device, the mobility indicator being a resultant of a combination of clinically relevant outputs, the method comprising:
obtaining mobility data by at least two monitors, disposed on different positions on at least one of the prosthesis or a body part of the amputee;
analysis of the mobility data; and
assessment of mobility indicator from the analyzed mobility data.

8. The method according to claim 7 further comprising suggesting the mobility indicator by a report tool.

9. The method of claim 7, wherein at least some of the mobility data is obtained as blind data.

10. The method of claim 7, wherein the assessment of the mobility indicator further comprises the use of user input data.
